(19) United States
(12) Patent Application Publication
(10) Pub. No.: US 2007/0055233 A1
(43) Pub. Date: Mar. 8, 2007

(54) Apparatus and method for repositioning fractured bone fragments using an arc shaped panel and half pins

(76) Inventor: Mark R. Brinker, Houston, TX (US)

Correspondence Address: GREENBERG TRAURIG, LLP, 77 WEST WACKER DRIVE, SUITE 2500, CHICAGO, IL, 60601-1732, US

(21) Appl. No.: 11/497,077

(22) Filed: Aug. 1, 2006

Related U.S. Application Data
(60) Provisional application No. 60/704,912, filed on Aug. 3, 2005.
(60) Provisional application No. 60/725,864, filed on Oct. 11, 2005.

(51) Int. Cl. A61B 17/00

(52) U.S. Cl. 606/54

(57) ABSTRACT

A system is disclosed for externally repairing fractured bones and facilitating alignment of displaced fractured bone segments without requiring use of an external ring fixator system and tension wires. The system comprises at least one panel member having a plurality of apertures extending from a first side to a second side of the panel member. At least two pin carriers are capable of being inserted into at least two of the plurality of apertures in the at least one panel member, the pin carriers, upon insertion into one of the plurality of apertures in the panel member, being longitudinally fixed relative to the aperture, but capable of rotation within the aperture. At least two half-pins are capable of insertion into the a pin carrier, following insertion of the one pin carrier into one of the plurality of apertures provided in the panel member, toward subsequent securement to a fractured bone segment. Rotation of a pin carrier causes an associated half-pin inserted therein to move longitudinally with respect to the panel member to, in turn, reposition the fractured bone segment affixed to the half-pin, relative to the panel member.

This application claims priority of the filing date of U.S. provisional patent application 60/704,912, filed 3 Aug. 2005 and U.S. provisional patent application 60/725,864, filed 11 Oct. 2005, the complete disclosures of which are hereby expressly incorporated herein by reference.

### BACKGROUND OF THE INVENTION

1. Field of the Invention

The present invention relates to stabilizing fractures and aligning displaced fractured bone fragments in a manner that readily allows for multiplanar half-pin fixation without the need for cumbersome and awkward bar-to-pin and pin-to-pin clamps used in a conventional unilateral external fixation system or the use of tensioned wires as used with a conventional ring-to-ring external fixation system structure. The present invention relates, in particular, to an apparatus providing for the quick and highly accurate alignment of fractured bone segments through the placement and precise adjustment of half-pins and blunt pins secured to an external planar member.

2. Background and the prior art

Various methods are currently available and are widely used by orthopedic surgeons to stabilize acute fractures and reduce displaced fractured bone fragments. Various of these prior art methods utilize external fixation devices. The currently practiced methods using unilateral external fixation systems are awkward and cumbersome and the currently practiced methods using ring-to-ring external fixation systems are technically demanding.

Unilateral External Fixation Systems

A typical prior art method for stabilizing an acute fracture utilizing a unilateral external fixation system incorporates external bars that, together with clamps, form a linear framework that supports a broken limb. The prior art unilateral external fixation system must be constructed and specifically tailored to each individual patient and is preferably positioned so as to overlie the point of fracture. Half-pins (as known to those skilled in the art) are positioned within the bone above and below the point of fracture and are connected to external bars using so called bar-to-pin clamps. In order to augment stability, multiple bars may be used and are connected to one another using so called bar-to-bar clamps. Stability may also be augmented by positioning the half-pins within the bone at various angles and within various planes so that the half-pins converge and diverge. This arrangement of half-pins inserted and secured to the bone segments is the so-called delta configuration.

Typical prior art unilateral systems substantially limit the surgeon's choices in regards to where half-pins may be positioned within the bone segments and some only allow half-pins to be placed in a linear fashion such that they are all parallel to one another. Other systems which allow for independent half-pin placement require cumbersome and awkward bar-to-pin clamps and pin-to-pin clamps in order to achieve the desirable delta configuration. The method to achieve this construction is slow and tedious.

To reduce a fracture and align a broken bone using a unilateral external fixation system a surgeon typically has to manually hold the bone segments in an aligned position while at the same time applying the external fixator. Some prior art methods allow for the incorporation of hinges or ball joints in order to allow for deformity correction. These hinges and ball joints are typically located at a fixed predetermined location along the length of the external fixation apparatus away from the apex of the fracture deformity. When there is a mismatch between the level of the hinge/ball joint and the level of the deformity, incomplete or incorrect movement of the bone segments occurs. These systems also do not permit the movement and reduction of displaced bone fragments through the manipulation of the half-pins which are anchored within the bone segments.

Ring-to-Ring External Fixator Systems

A typical prior art method for treating a displaced fracture utilizes an external fixator system which incorporates a plurality of rings that together with supporting rods, brackets, nuts and bolts, form a framework surrounding and supporting a broken limb. The prior art external ring fixator must be constructed and specifically tailored to each individual patient and is preferably positioned so as to overlie the point of fracture. The rings are typically spaced apart and held together in a rigid assembly by a series of threaded rods which form the framework around the limb. Rings above and/or below the point of facture each typically support tensioned wires and half-pins that function to effectively secure the external ring framework to the upper and lower fractured bone fragments. Special techniques have been devised to permit the surgeon to reduce the fracture using the tensioned wires but no accurate methods for bone reduction are available in the typical prior art.

To reduce a fracture and align a broken bone a surgeon typically passes Olive Wires through the skin and bone at positions above and below the point of the fracture. Olive Wires are manually manipulated to pull facture fragments into alignment in order to correct frontal plane angular deformities. The arched wire technique is often used to correct sagital plane angular and translational deformities. In both cases it is often a cumbersome procedure to tension a wire and maintain the desired tension and position while securing the free ends of the wire to the ring.

It can be appreciated that the fractured bone fragments are moved into a desired aligned positioned with respect to one another by the force exerted on the bone by the wire. The surgeon's skill and experience determines where along the ring the wire should be affixed and how tight the wire needs to be drawn in order to move the bone fragment to a desired position.

An x-ray is taken to verify the position of the fractured bone fragments and the process is then repeated, sometimes again and again, until the desired position is achieved. The process is very tedious and time consuming. In fact, one significant disadvantage with this prior art method is that each successive repositioning step tends to displace the prior reduction with the surgeon adjusting one offset after another, sometimes never getting the fracture accurately reduced. In addition, oblique plane deformities which are neither purely in the frontal nor sagital plane are typically reduced by using these techniques executed in sequence, first reducing the fracture in the frontal plane and then reducing the fracture in the sagital plane.

Moreover, each adjustment requires that one or both ends of the wire be freed from its post such that tension on the wire is effectively released, notwithstanding the surgeon's attempt to maintain tension while making an adjustment. This prior art method is generally a gross reduction maneuver that lacks the necessary precision and accuracy to optimally correct a displaced fracture.

In addition, the surgeon is required to construct a cumbersome frame surrounding the broken limb.

Accordingly it is a desirable characteristic of the invention to provide for the precise reduction of a bone fracture and achieve precise alignment with a direct rigid connection to the bone and without the use of wires or requiring the assembly and use of a complex multi-piece external supporting structure.

It is a further characteristic of the present invention to provide for the precise reduction of a bone fracture in a quick and efficient manner which omits trial and error.

These and other characteristics of the present invention will become apparent to one of skill in the art having the present disclosure before them.

### SUMMARY OF THE INVENTION

A system is disclosed for externally repairing fractured bones and facilitating alignment of displaced fractured bone segments without requiring use of an external ring fixator system and tension wires. In one embodiment the invention includes at least one panel member having a plurality of apertures therein, extending from a first side of the at least one panel member through to a second side of the at least one panel member. At least one pin carrier capable of being inserted into at least one of the plurality of apertures in the at least one panel member is provided. The at least one pin carrier, upon insertion into at least one of the plurality of apertures in the at least one panel member, is longitudinally fixed relative to the aperture, and capable of rotation within the aperture. At least two half-pins being capable of insertion into the at least one pin carrier, following insertion of the at least one pin carrier into one of the plurality of apertures provided in the panel member, provides securement to a fractured bone segment.

In one embodiment, the at least one panel member is further configured to accommodate at least one outrigger member capable of being removably affixed to the panel member, the outrigger member is capable of accepting receipt of a half-pin toward securing the outrigger member to a fractured bone segment. A second one of the at least two half-pins is capable of insertion into one of another pin carrier inserted into another one of the plurality of apertures in the at least one panel member and at least one outrigger affixable to the at least one panel member. The at least two half-pins serve, in part, to support the at least one panel member overlying the fractured bone segments. Rotation of a pin carrier causes an associated half-pin inserted therein to move longitudinally with respect to the at least one panel member to, in turn, reposition the fractured bone segment affixed to the half-pin, relative to the at least one panel member.

In a disclosed embodiment, wherein the plurality of apertures provided in the panel member are formed in a plurality of symmetrical rows and columns providing multiple points at which a half-pin can be inserted and positioned relative to the bone segments to be aligned. The at least one pin carrier is substantially cylindrical in shape and includes at least one annular ring formed on an outer facing surface thereof. The pin carrier includes a helical thread formed on an inner facing surface thereof which thread cooperates with threads formed on at least a portion of the at least one half-pin, and includes a control surface to facilitate rotation of the pin carrier, following insertion into one of the plurality of apertures in the panel member. In one embodiment, the control surface comprises a knob.

In a preferred embodiment, each of the plurality of apertures within the panel member is formed with at least one annular ring on an inner surface and operably configured for cooperation with an outer facing annular ring of the pin carrier. In one embodiment, the panel member may be further provided with apertures dimensioned and threaded to directly accept at least one half-pin, without insertion of an intervening pin carrier. In a preferred embodiment, the panel member is arc shaped, and is further operably configured to generally conform to the shape of a limb surrounding fractured bone segments. The panel member may further include a roller bearing joint positioned in at least one aperture and capable accepting a pin carrier permitting a pin carrier, and in turn, a half-pin to be secured to a fractured bone segment at an angle relative to the surface of the panel member.

In one illustrated embodiment, the present invention includes at least one outrigger member capable of being removably affixed to the a panel member and capable of accepting receipt of a half-pin toward securing the outrigger member to a fractured bone segment. The length of the outrigger, upon attachment to an upper or lower edge region of the at least one panel member, may be alternatively increased or decreased as necessary to position the panel member in an overlying orientation with respect to the fractured bone segment.

The panel member may further comprise two panel or more members, secured together to form a single reconfigurable composite panel member capable of supporting pin carriers and, in turn, half-pins in a manner that permit the half-pins to be secured to the fractured bone segments in a plurality of planes, such as in a delta configuration. A first panel member may incorporate a groove formed on a lower facing edge thereof and a second panel member may incorporates a tongue formed on an upper facing edge thereof, to thereby facilitate joining the first panel member to the second panel member. A first panel member segment may thus be rotated and secured relative to a second panel member segment.

The invention in one embodiment may include a first panel member incorporating a threaded aperture formed on a lower facing edge and a second panel member incorporating a threaded aperture formed on an upper facing edge to facilitate joining one panel member to the another panel member using a threaded rod.

In an alternative further embodiment of the present invention, the invention is disclosed as a system comprising at least one panel member having a plurality of apertures extending from a first side to through to a second side; at least two pin carriers, capable of being inserted into at least two of the plurality of apertures in the panel member and upon insertion into at least apertures in the panel member are longitudinally fixed relative to the aperture, but capable of rotation within the aperture; at least one half-pin and one blunt-pin each capable of insertion into the a pin carrier, whereupon rotation of a pin carrier causes an associated half-pin inserted therein to move longitudinally with respect to the panel member to, in turn, reposition a fractured bone segment, relative to the panel member.

A further embodiment of the present invention comprises an automated computer assisted system for aligning displaced fractured bone fragments including , a panel member having a plurality of apertures therein and affixable to the displaced fractured bone fragments by a plurality half-pins with at least a first half-pin positionable above the point fracture and a second half-pin positionable below the point of fracture, the panel member, in turn, secured to the upper and lower fractured bone fragments by half-pins passed through the skin and into underlying bone and at least one pin carrier positioned in an aperture and configured to accept telescopic receipt of a half-pin, the rotation of the pin-carrier serving to adjust the position of the half-pin with respect to the panel member toward facilitating alignment of the displaced fracture bone fragments. A computer controlled electronic servo motor is operably connected to each pin carrier for precisely and independently adjusting the position of a half-pin positioned therein. A computer controlled x-ray imaging and control system creates and analyzes an electronic image, determining the relative displacement of the fractured bone fragments and signaling the controlled electronic servo motors to adjust the position of each pin carrier toward aligning the displaced fractured bone fragments without manual intervention. The automated computer assisted system may further include reference wires inserted into the fractured bone fragments to enhance detection of the bone fragments by the x-ray imaging and control system and establish the initial position of the fractured bone fragments.

The computer controlled x-ray imaging and control system analyzes the initial position of the fractured bone fragments, computes the distance and sequence in which each bone fragment must be moved in order to properly restore alignment and signals the controlled electronic servo motors in a coordinated manner to move in the proper sequence toward articulating the fractured bone fragments into proper alignment. Back pressure sensors may be provided and associated with one or more pin carriers and/or half-pins toward monitoring the force exerted by the half-pin upon the limb and providing an alarm signal to the x-ray imaging and control system.

The present invention further is disclosed as a method for aligning displaced fractured bone fragments, the method comprising the steps of: providing a panel member for affixation to the fractured limb with at least a first half-pin positionable above the fracture and a second half-pin positionable below the fracture; affixing at least one pin carrier to the panel member positioned adjacent to the fracture; inserting at least one half-pin into at least one pin carrier and affixing the half-pin to a fractured bone fragment; adjusting the pin carrier to reposition the associated half-pin to reposition the underlying bone fragment affixed thereto to reduce the fracture and align the bone fragments; whereby the fracture is reduced and bone fragments aligned without the surgeon having to use external ring fixator system and tension wires to reposition misaligned bone fragments.

### BRIEF DESCRIPTION OF THE DRAWINGS

FIG. 1 of the drawings illustrates a front elevation view of one embodiment of an arc fixator panel according to the present invention having an outrigger affixed to the lower edge thereof;

FIG. 2 of the drawings illustrates a side elevation view of the outrigger of FIG. 1 in a semi-retracted state showing the repositionable disc member thereof;

FIG. 3 of the drawings illustrates a side elevation view of the outrigger of FIG. 1 shown in a fully extended state;

FIG. 4 of the drawings illustrates a side cross-sectional view of a portion of the arc fixator panel specifically showing the steps of inserting a half-pin through a pin carrier inserted into apertures formed in the arc panel and into a bone fragment;

FIG. 5 of the drawings illustrates a side elevation view of a pin carrier according to the present invention which is inserted into apertures provided in the arc panel and capable of telescopically accepting a half-pin;

FIG. 6 of the drawings illustrates a side elevation view of a pin carrier according to the present invention showing it being manually compressed toward facilitating insertion into an aperture provided in the arc panel and capable of accepting insertion of a half-pin;

FIG. 7 of the drawings illustrates a perspective view of a pin carrier specifically showing the threaded interior surface and the outer surface bearing a series of annular rings;

FIG. 8 of the drawings illustrates a side elevation view of the arc panel in cooperation with an outrigger of the present invention configured to lengthen broken bone fragments toward aligning same;

FIG. 9 of the drawings illustrates a side elevation view of the arc panel in cooperation with an outrigger of the present invention specifically illustrating the lengthening of broken bone fragments;

FIG. 10 of the drawings illustrates the rotation of a pin carrier toward alternatively retracting and advancing a half-pin which serves to move the bone fragment attached to the distal end of the half-pin;

FIG. 11 of the drawings illustrates a top plan cross-sectional view of the arc fixator panel positioned adjacent a broken limb and the placement of three half-pins in a manner which secures the broken bone fragment in a stable manner using a delta half-pin configuration;

FIG. 12 of the drawings illustrates a front elevation view of an alternative embodiment of an arc fixator panel according to the present invention specifically showing an arc panel comprising four separate segments;

FIG. 13 of the drawings illustrates a front elevation view of a further embodiment of an arc fixator panel according to the present invention specifically comprising an assembly of four separate segments and ability to reposition the upper-most segment with respect to the lower three segments;

FIG. 14 of the drawings illustrates a front elevation view of a still further embodiment of an arc fixator panel according to the present invention specifically comprising four separate segments and the ability to reposition the upper-most segment with respect to the three lower segments and also rotate the lower two segments with respect to the upper two segments;

FIG. 15 of the drawings illustrates a side elevation view of the arc panel according to FIG. 13 illustrating the use of the present invention to lengthen broken bone fragments toward alignment of same;

FIG. 16 of the drawings illustrates a side elevation view of the arc panel according to FIG. 13 specifically illustrating the repositioning of the upper-most segment to separate the broken bone fragments toward the alignment of same;

FIG. 17 of the drawings illustrates a side elevation view of the arc panel according to FIG. 13 specifically illustrating the advancing of the half-pin toward translating the broken bone fragments into alignment;

FIG. 18 of the drawings illustrates a side elevation view of the arc panel according to FIG. 13 specifically illustrating the reduction of fractured bone segments;

FIG. 19 of the drawings illustrates a side cross-sectional view of a roller bearing joint positioned within an aperture formed in the arc panel toward permitting a half-pin to be positioned at an angle with respect to the plane formed by the arc panel;

FIG. 20 of the drawings illustrates a side cross-section view of the roller bearing joint according to the present invention;

FIG. 21 of the drawings illustrates a side elevation view of the arc panel according to FIG. 12 specifically illustrating the use of the present invention to reduce a bone fracture with angulation;

FIG. 22 of the drawings illustrates a side elevation view of the arc panel according to FIG. 12 specifically illustrating the reduction of fractured bone segments;

FIG. 23 of the drawings illustrates a side elevation view of the arc panel according to FIG. 13 specifically illustrating the use of the present invention to reduce a bone fracture with rotation;

FIG. 24 of the drawings illustrates a side elevation view of the arc panel according to FIG. 13 specifically illustrating the reduction of fractured bone segments;

FIG. 25 of the drawings illustrates a top plan cross-sectional view of the arc fixator panel positioned adjacent a broken limb and the placement of one half-pin secured to the broken bone fragment in a stable manner and one blunt pin positioned in contact with but not affixed to the broken bone fragment;

FIG. 26 of the drawings illustrates the rotation of a pin carrier toward advancing a blunt pin into contact with the external surface of the bone fragment;

FIG. 27 of the drawings illustrates the rotation of a pin carrier to the degree that the blunt pin has been advanced such that the distal end of the pin is in contact with the external surface of the bone fragment; and

FIG. 28 of the drawings illustrates a further embodiment of the present invention comprising an automated computer assisted system for aligning displaced fractured bone fragments.

### DETAILED DESCRIPTION OF THE DRAWINGS

While the present invention is susceptible of embodiment in many different forms, there are shown in the drawings and will be described in detail, several specific embodiments, with the understanding that the disclosure herein is to be considered as an exemplification of the principles of the present invention and is not intended to limit the invention to the embodiments illustrated.

The present invention is illustrated in the context of a fractured leg bone with the understanding the present invention has application in many other situations. For example, the present invention may be used to move and align deformities in long bones, joints and other bone structures.

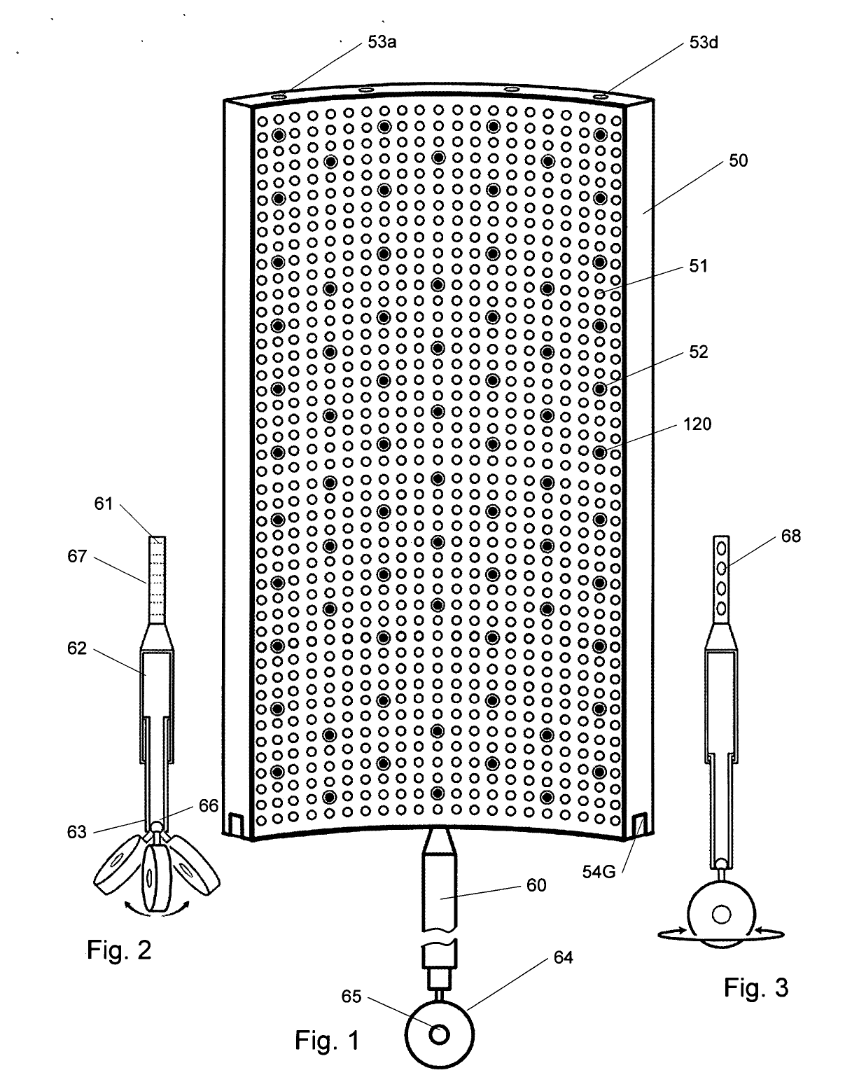

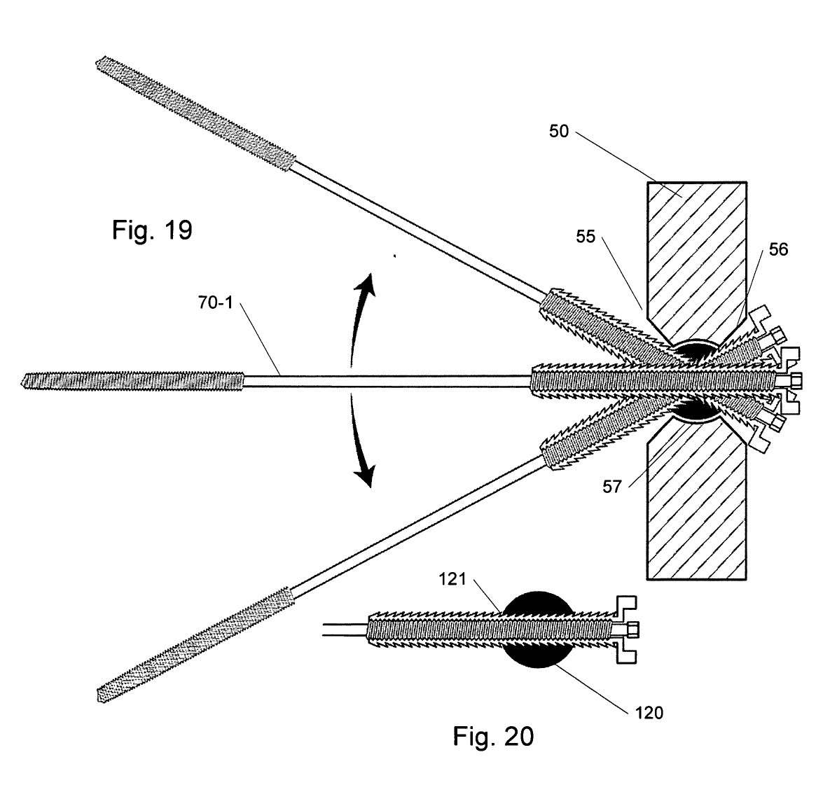

FIG. 1 of the drawings illustrates arc fixator panel 50 shown in cooperation with outrigger member 60. Arc fixator panel 50 is illustrated having a plurality of apertures 51 formed therein and arranged in symmetrical rows and columns. Various apertures designated with reference numeral 52 are shown having roller bearing joint 120 inserted therein. The purpose of apertures 51 and roller bearing joints 120 is described in connection with the description of FIGS. 19-22. Arc fixator panel 50, alone and/or in cooperation with outrigger member 60, serves to take the place of the cumbersome and complex unilateral external fixation frameworks which are typically required to be assembled in order to support and carry bars and half-pins as well as the cumbersome and complex circular ring external fixation frameworks which are typically required to be assembled in order to support rings and tension wires that are used to draw fractured bone segments into alignment and maintain such alignment, towards permitting the fracture to heal. Arc panel 50 is shown in the illustrated embodiment as spanning an arc of approximately 120 degrees, though it is contemplated that panels of a greater or lesser angular span, such as 180 degrees or 90 degrees may be used to achieve the desired objective of the present invention as disclosed and described herein. Indeed, arc panels 50 could be joined together side-by-side to completely surround a limb in a 360 degree circle. Arc panel 50 may be fabricated of virtually any stiff material suitable for use in an operating room or battlefield environment.

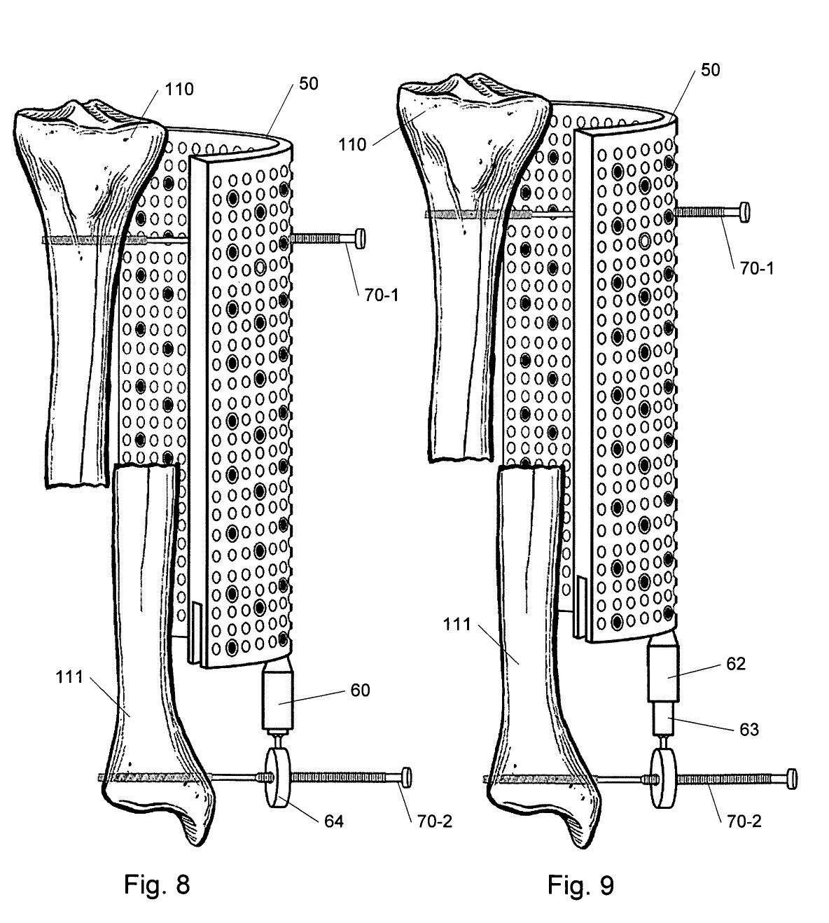

As illustrated in FIGS. 2 and 3, outrigger 60 comprises upper and lower members 62 and 63, respectively, which are adjustable with respect to one another, in that upper member 62 telescopically receives lower member 63. A series of threads on the inner surface of upper member 62 and on the outer surface of lower member 63 engage with one another, such that rotation of the upper outrigger member with respect to the lower outrigger members serves to alternatively increase or decrease the overall length of outrigger member 60. Disc 64 emanates from the lower portion 63 of outrigger member 60 and serves to accept insertion of a half-pin. Aperture 65 formed within disc 64 is preferably threaded and serves to engage with the outer threads of half-pin 70-2, as described below. (As illustrated in FIG. 8, arc panel 50 is secured to the upper displaced fractured bone segment 110 using half pin 70-1. Lower displaced fractured bone segment 111 is shown secured to arc panel 50 via outrigger 60 and half-pin 70-2.)

Outrigger 60 is shown attached to the bottom facing edge of arc panel 50. Outrigger 60 may be removably affixed to panel 50 using a number of mechanisms. FIG. 2 illustrates pin 61 emanating from upper member 62, which includes threads 67 that engage with threads formed into an aligned aperture 53b formed into the bottom edge of panel 50. FIG. 3 illustrates an alternative embodiment where tip 61 emanating from upper member 62 includes apertures 68. Tip 61 may be inserted into an aligned aperture along the bottom edge of panel 50 and secured in place with a screw inserted into an aperture 51 in panel 50 overlying aperture 68. Outrigger 60 may be provided in various lengths to permit lower half-pin 70-2 to be affixed to the lower fractured bone segment a distance away from the fracture without having to provide an overly long arc panel 50.

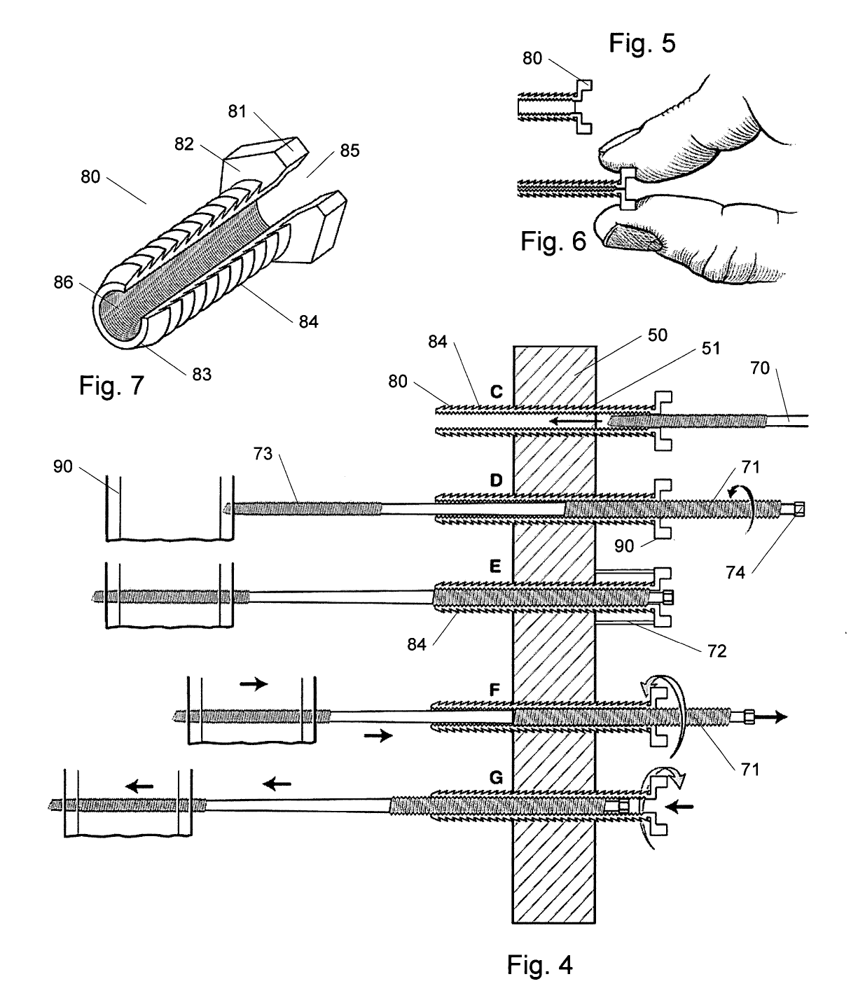

FIGS. 4-7 of the drawings illustrate a particularly novel aspect of the present invention which serves to replace the cumbersome prior art use of tension wires and complex external rings and frameworks to align fractured bone segments. As described hereinbelow, the cooperation of a half-pin, a pin carrier, an arc fixator panel and the apertures formed therein serve to permit a surgeon to align fractured bone fragments with the utmost precision and convenience in a manner heretofore unknown.

FIG. 4 of the drawings illustrates a cross-section of a portion of arc fixator panel 50. To facilitate the present disclosure the steps of inserting a half-pin through a pin carrier and into a bone fragment, and displacement of the bone fragment through rotation of the pin carrier are illustrated therein by reference letters C through G. For clarity, FIG. 4 shows only the broken bone fragment and omits showing any surrounding tissue or skin with the understanding that arc panel 50 may remain spaced away from the external skin surface.

As shown in relation to reference letter C, pin carrier 80 is inserted into an aperture 51 preformed into arc panel 50. Pin carrier 80 is illustrated in FIGS. 5-7 and comprises, in one embodiment, a flexible insert formed having groove 85 extending longitudinally along one side towards permitting the pin carrier to be deformed by manual force as illustrated with reference to FIG. 6. In its normal expanded shape (illustrated in FIGS. 5 and 7), pin carrier 80 has, at one end, a head (or hat) portion 81, having an inner facing shoulder surface 82. At its opposite end, tapered portion 83 serves to assist and facilitate the insertion of pin carrier 80 into an aperture 51. The outer surface of pin carrier 80 includes a series of annular ridges 84 which cooperate with a corresponding series of annular grooves and ridges formed into the interior surface of each aperture 51. The purpose of annular rings 84 is to cooperate with the aforementioned grooves and ridges formed within apertures 51, such that when pin carrier is inserted, it may be rotated therein without pin carrier advancing or retracting as would be the case if helical threads were used. The inner surface of pin carrier 80 is however formed with threads 86 which cooperate with the threads 71 provided in the proximal end of half-pin 70. An alternative embodiment of pin carrier 80 may omit groove 85.

As shown by reference C, pin carrier 80 is inserted into aperture 51 such that head 81 does not completely abut the outer facing surface of arc panel 50. Half-pin 70 is shown having a threaded end 73 which is inserted into the open end of head 81 of pin carrier 80. The purpose of threads 71 are to tap into bone fragment 90 and secure half-pin 70 thereto. It will be appreciated that the surgeon may use a drill guide to drill a pilot hole into bone fragment 90 along the longitudinal axis of pin carrier 80 to facilitate insertion of half-pin 70 into bone 90. Alternatively, self-tapping half-pins may be used.

It can be seen with reference to letter D that half-pin 70 has been rotated clockwise such that it has advanced through pin carrier member 80 to the degree that threads 73 at the distal end thereof engage with and begin to tap into bone fragment 90.

Reference E illustrates half-pin 70 fully inserted into bone fragment 90, such that it is completely set into the bone and retained within arc panel 50 by pin carrier 80. Accordingly, it can be appreciated with reference to letter F that clockwise rotation of pin carrier 80 will serve to cause half-pin 70 to be drawn outward from the panel 50 and, in turn, the fractured bone fragments such that the fractured bone fragment is displaced from its initial position. Conversely, as shown with respect to reference G, it can be appreciated that a counter-clockwise rotation of pin carrier 80 will cause half-pin 70 to advanced inwardly toward the limb with respect to the arc panel 50 such that the fractured bone fragment is displaced from its initial position.

It can be appreciated that FIGS. 4-7 illustrate but a single embodiment of the cooperation of pin carrier 80 and apertures 51 formed into the cross-section of arc panel 50. For example, the annular rings formed on the external surface of pin carrier 80 are angled such that they facilitate the insertion of pin carrier 80 into an aperture 51 and yet restrict removal of the pin carrier from aperture 51 by virtue of their angular formation and co-operation with the corresponding annular grooves formed within aperture 51.

It can be appreciated that depending upon the location, muscle tone, tissue, nature of the injury, etc. rotation of the pin carrier may cause pin carrier to be pulled further inward together with half-pin 50, as opposed to remaining within aperture 51 and merely advancing the half-pin. Accordingly as shown in reference E, spacer 72 may be provided and positioned between the outer facing surface of panel 50 and shoulder 82 of pin carrier 80, thereby restricting any further inward movement of the pin carrier. Alternatively, annular rings 84 could be formed with no angle with respect to a corresponding cooperating series of rings, grooves and ridges such that inward and outward movement of pin carrier with respect to arc panel 50 is equally restricted.

FIG. 8 of the drawings illustrates one embodiment of the present invention. Upper and lower fractured bone segments 110 and 111 are shown requiring lengthening in order to then translate and align the fractured bone segments. Arc panel 50 is initially affixed to the upper bone fragment 110 using half pin 70-1. For the sake of clarity, pin carrier 80 is not shown. Lower bone fragment 110 is secured to outrigger 60 which is, in turn, affixed to arc panel 50 as described above. Rotation of the lower outrigger member 63 with respect to upper outrigger member 62 serves to extend the overall length of outrigger 60 towards separating bone fragments 110 and 111, as illustrated in FIG. 9.

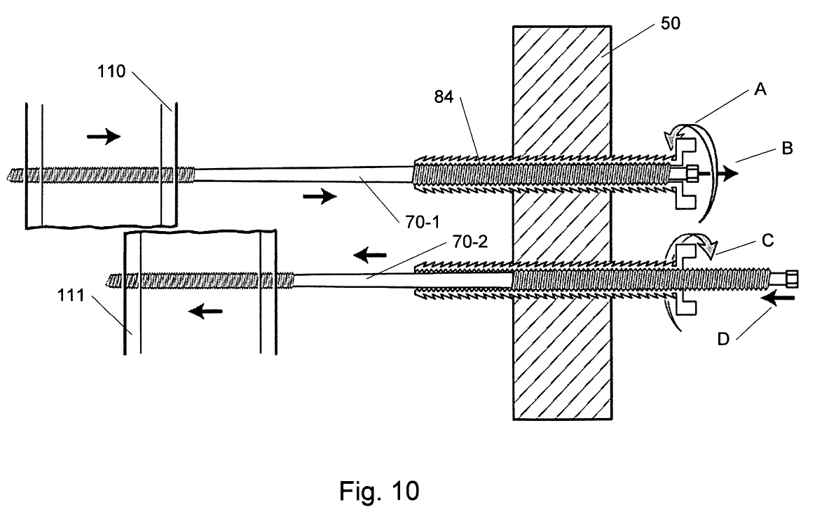

It can be appreciated with respect to FIG. 9 that translation of fractured bone segments can be addressed through the use of the present combination of arc panel 50, half-pins 70 and pin carriers 80. An illustration of this basic concept is provided in FIG. 10. Half-pin 70-1 is shown affixed to the upper bone fragment 110 whereby a clockwise rotation of pin carrier 80 causes half-pin 70-1 to be withdrawn from the arc panel 50 which, in turn, draws the fractured bone segment 110 to the right as illustrated. Conversely, the half-pin 70-2 shown affixed to lower fractured bone segment 111 likewise can be manipulated through rotation of its cooperating pin carrier 80 whereby a counter-clockwise rotation of pin carrier 80 serves to advance half-pin 70-2 inward with respect to arc panel 50 and thereby move the fractured bone fragment 111 to the left, as illustrated by the corresponding arrow.

It can be appreciated that the pin carriers 80 can be rotated as necessary to effect the required degree of translation of bone fragments. It is further envisioned that pin carriers 80 may be rotated through the manual rotation of heads 81 formed thereon configured to accept an external tool, such as a socket, which may be rotated manually or by motor-driven apparatus under the control of the attending surgeon. Moreover, the spacing of the threads formed on the inner surface of pin carriers 80 and outer surface of half-pin 70 may be of various configurations that provide finer or coarser degrees of movement with respect to each 360 degree rotation of pin carrier 80, such that greater or fewer turns are required to move a half-pin 70 a given distance and, in turn, the bone fragment affixed to the distal ender thereof. As described below, it can be further appreciated that the mechanized rotation of the pin carriers can automated and under control of a computer-driven system, which, together with a fluoroscopy system, can automate the process of reducing fractured bone fragments.

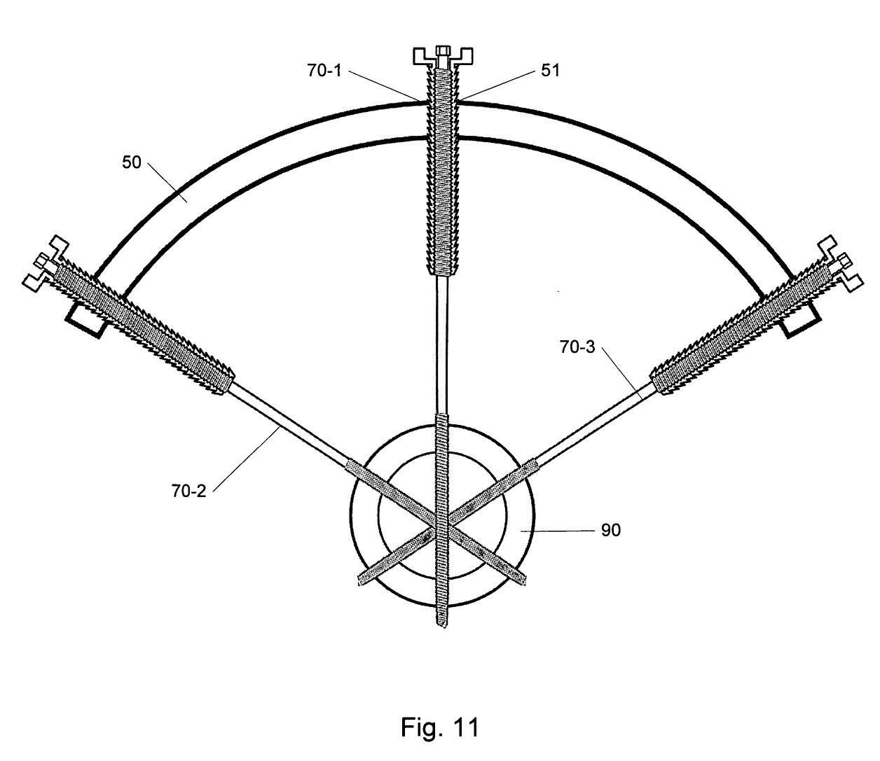

One advantage of the present invention is the ability to provide a highly stable fixator without the need for the surgeon to assemble a complex, multi-part external fixator as presently utilized in the prior art. As illustrated in FIG. 11, the arc panel 50 and the plurality of apertures 51 formed therein permit a plurality of half-pins 70-1, 70-2 and 70-3 to be driven into and affixed to the fractured bone segments from a plurality of directions. The positioning of half-pins in multiple planes serves to effectively secure the bone fragment to arc panel 50 in a rigid and highly stable manner comparable, if not superior to that provided by prior art external fixator systems set up in a delta configuration. In the example illustrated, three half-pins are used to secure bone fragment 90 to surrounding arc panel 50. The half-pins are shown equally spaced approximately 60 degrees from one another. It is of course contemplated that spacing may be dictated by the specific bone that has been fractured and the desire to secure a half-pin to a specific “side” or bone surface. The plurality of apertures 51 provided in arc panel 50 facilitate and indeed expedite the placement of half-pins in such situations. Of course, it is also contemplated that half-pins may be inserted into apertures in different rows within panel 50 to provide vertical separation of the half-pins in the bone structure in order to provide an appropriate and secure anchorage, without unduly damaging and/or weakening the fractured bone segment.

It is deemed further within the scope of the present invention that half-pins could be used to secure the bone fragments to arc panel 50 without use of pin carriers. Such an application would be dictated when a temporary repair is needed, such as in advance of impending surgery. In such case, larger diameter half-pins could be used which include self-tapping threads that can cut into and engage with apertures 51. Alternatively, arc panel 50 could be provided with additional or select apertures which are dimensioned and threaded to accept the same half-pins as used in association with pin carriers 80.

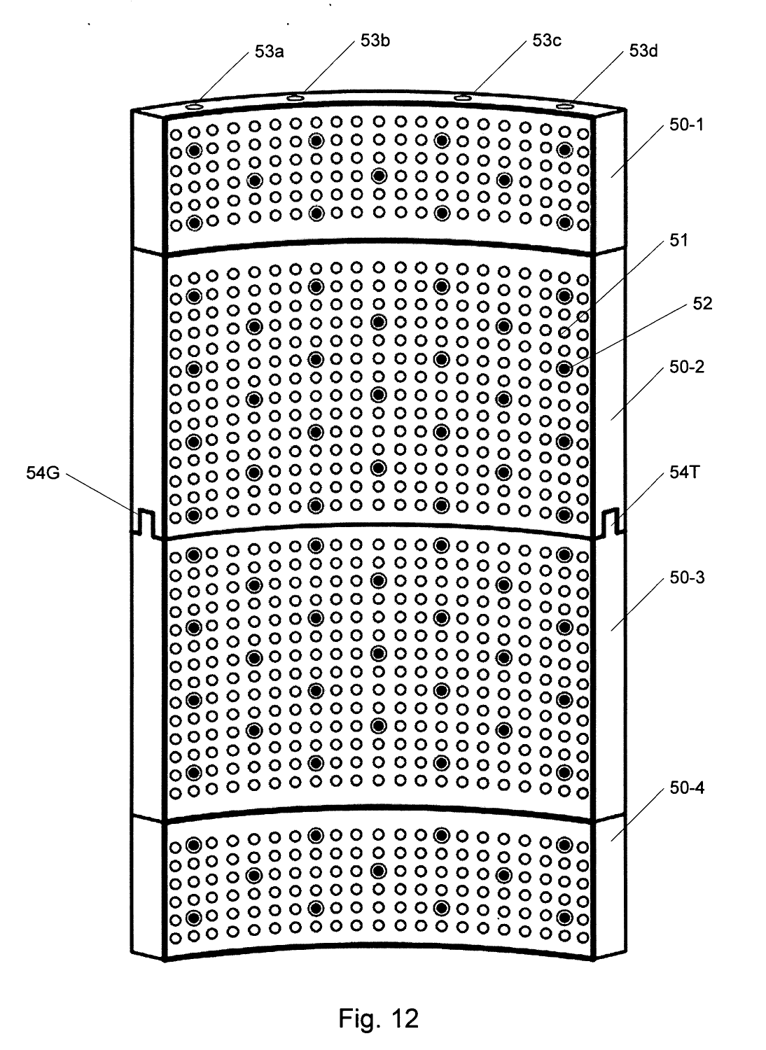

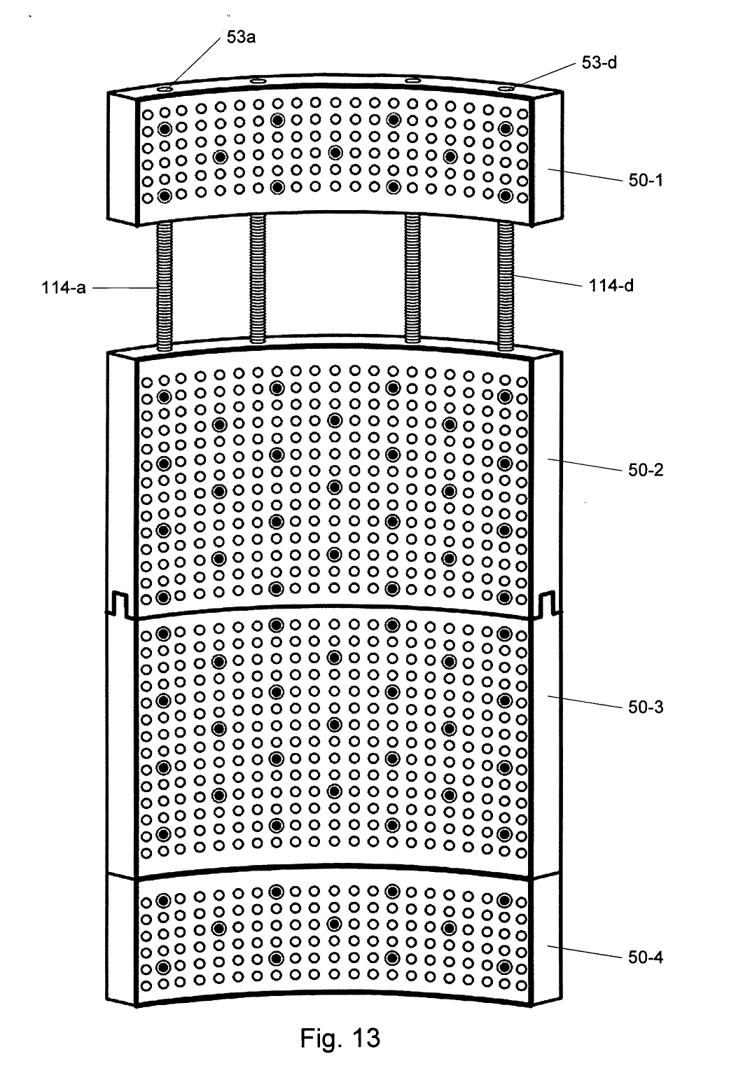

FIG. 12 of the drawings illustrates an alternative embodiment of arc panel 50. In the embodiment illustrated, the arc panel 50 is composed of four segments stacked on top of one another. While the upper and lower-most segments are shown as each having six rows of apertures and the inner two segments each having 14 rows of apertures, segments of different sizes and indeed different spacing of apertures is of course, contemplated. Accordingly, the required numbers of segments may be pre-assembled, depending upon the nature and location of the fracture or fractures which are sought to be addressed by the present invention. FIG. 13 of the drawings illustrates the extension of uppermost segment 50-1 with respect to the lower three segments. In the embodiment illustrated, four threaded rods 114 a-d connect uppermost segment 50-1 to segment 50-2. These threaded rods are inserted into and through segments via four threaded apertures 53 a-d formed in uppermost edge of the upper segment 50-1. The same mechanism for connecting lowermost segment to the segment immediately thereabove may be used. It is envisioned that connectors other than threaded rods are deemed within a scope of the present invention towards joining segments to one another.

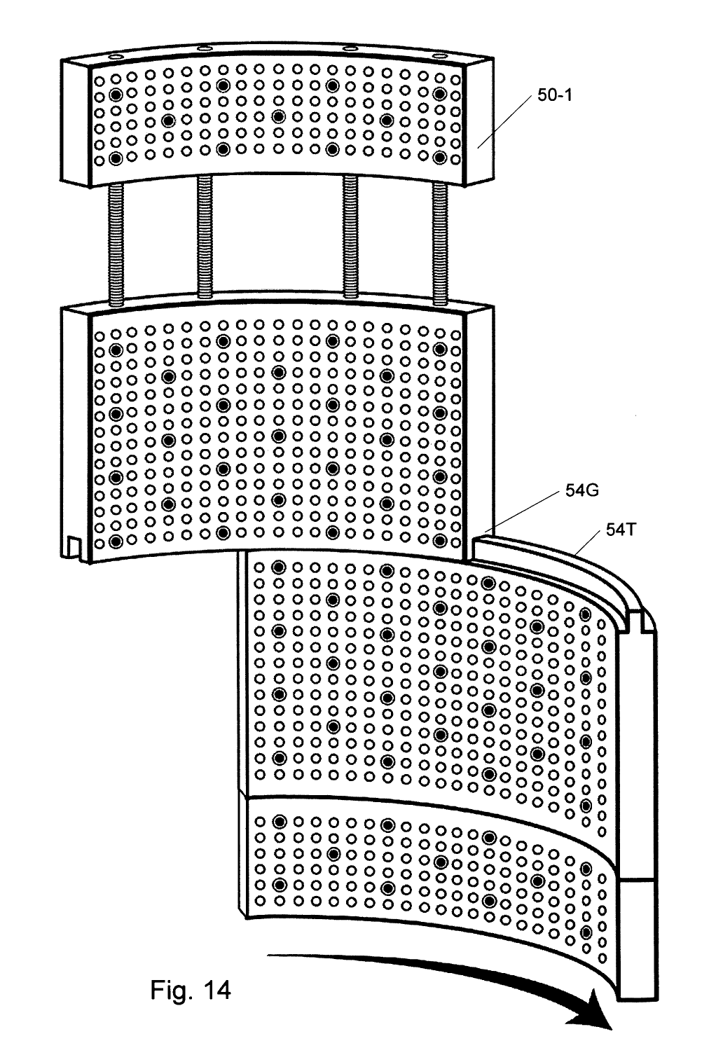

Two inner segments 50-2 and 50-3 are connected to one another via a tongue-and-groove arrangement. While different mechanisms are contemplated to join the two together, one may simply insert screws (not shown) into one or more apertures in the lower-most row, such that the screw pierces the groove and abuts against the underlying tongue to affix the two segments together. As appreciated in FIG. 14, the tongue-and-groove arrangement of the two illustrated segments permits upper two segments 50-1 and 50-2 to be rotated with respect to lower two segments 50-3 and 50-4, respectively, toward reducing a fracture requiring rotation, as further described below.

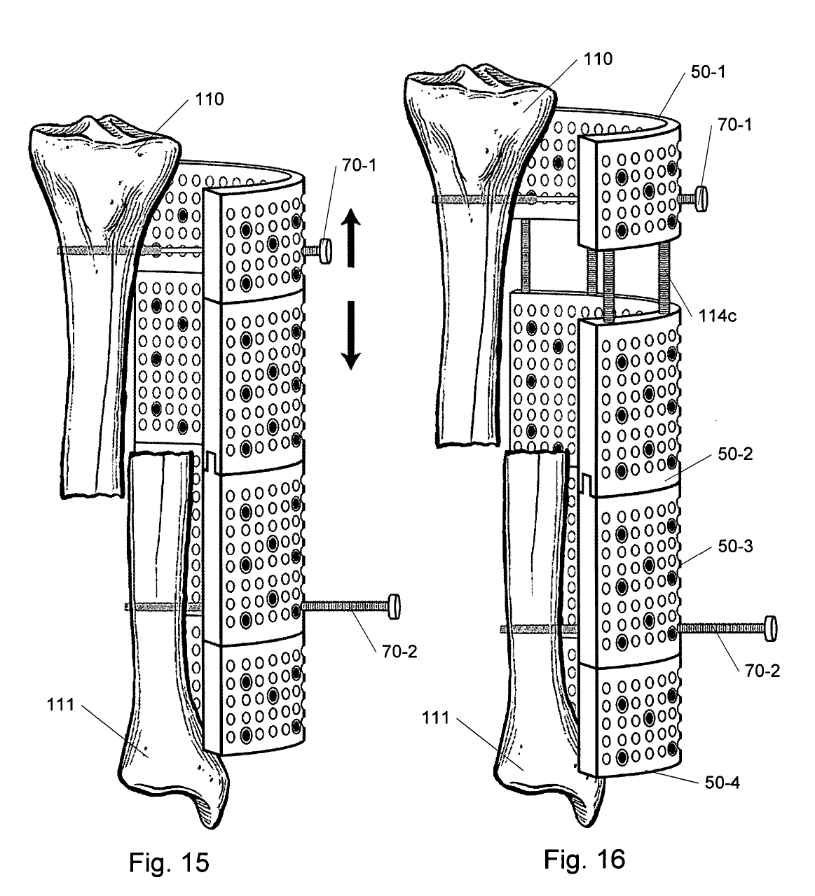

FIG. 15 of the drawings illustrates the use of the present invention to correct a fracture which requires lengthening of the limb to be able to translate the fractured bone segments. As illustrated in FIG. 15, four-segment composite arc panel 50 is used to correct the fracture. Upper fractured bone segment 110 is shown affixed to uppermost arc segment 50-1 via pin 70-1. Lower fractured bone segment 111 is shown affixed to the third bone arc segment 50-3 via pin 70-2, according to the prior description. Lengthening of the bone fragments with respect to one another is accomplished via movement of uppermost arc segment 50-1 with respect to arc segment 50-2 immediately below, as illustrated in FIG. 16. Access to threaded rods 114a-d is provided via apertures 53a-d formed in the upper-most edge of uppermost segment 50-1. It is contemplated that rods 114 may have a recess formed in their end to accept receipt of a tool toward rotating the rods and gradually adjusting the separation of the segments. Alternatively, the rods may each include a head portion which remains externally accessible for manual adjustment. As described in connection with half-pins 70, a motorized computer controlled system may be secured to each rod toward the automatic adjustment of same.

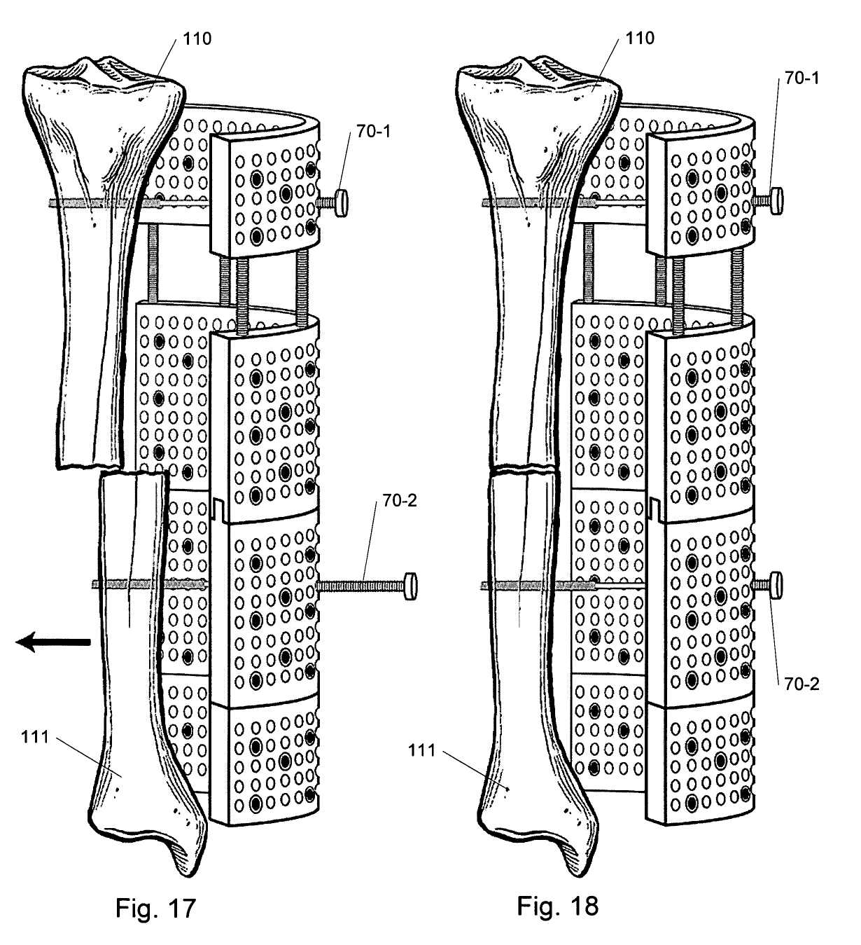

The step of translating the two fractured bone segments into alignment is illustrated with respect to FIGS. 17-18. In the illustrated embodiment, the pin carrier 80 (not shown) associated with pin 70-2 would be rotated counter-clockwise to cause lower bone fragment 111 to move to the left, as the figure is seen by the viewer. In the embodiment illustrated, it can be appreciated that the displaced fracture can be reduced and alignment of the two fractured bone segments accomplished merely through the manipulation of lower pin 70-2 and, specifically, its associated pin carrier. Of course, it is contemplated that multiple half pins may be moved by manipulation of their respective pin carriers towards accomplishing the necessary alignment, particularly in view of the construction of FIG. 11. FIG. 18 illustrates completed reduction of a fracture requiring translation of one fractured bone segment with respect to the other in order to align the two.

FIG. 19 of the drawings illustrates a side cross-sectional view of roller bearing joint 120 positioned within an aperture 52 formed in the arc panel 50 toward permitting a half-pin 70 to be positioned at an angle with respect to the plane formed by the arc panel 50. Roller bearing joint 120 comprises a generally spherical shaped element having an aperture 121 formed therein capable of receiving a pin carrier 80. Aperture 121 has a series of ridges and grooves formed therein similar to the inner surface of apertures 51 toward cooperating with the annular rings formed on the outer surface of pin carriers 80. As pictured, apertures 52 differ from apertures 51 inasmuch as each includes a tapered opening 55 and exit 56 and a generally concave region 57 formed at the center point. The generally spherical shaped bearing 120 is formed of a substantially rigid material yet having sufficient elasticity to permit it to be compressed to a degree to permit its insertion into aperture 52 and retained within the concave region therein.

It will be appreciated that the spherical shaped bearing joint permits a pin carrier and half-pin to be angled relative to the axis perpendicular to the surface of arc panel 50. As illustrated, and as further subject to the dimensions of the various components, a half-pin may be angled as much as approximately 30 degrees from perpendicular.

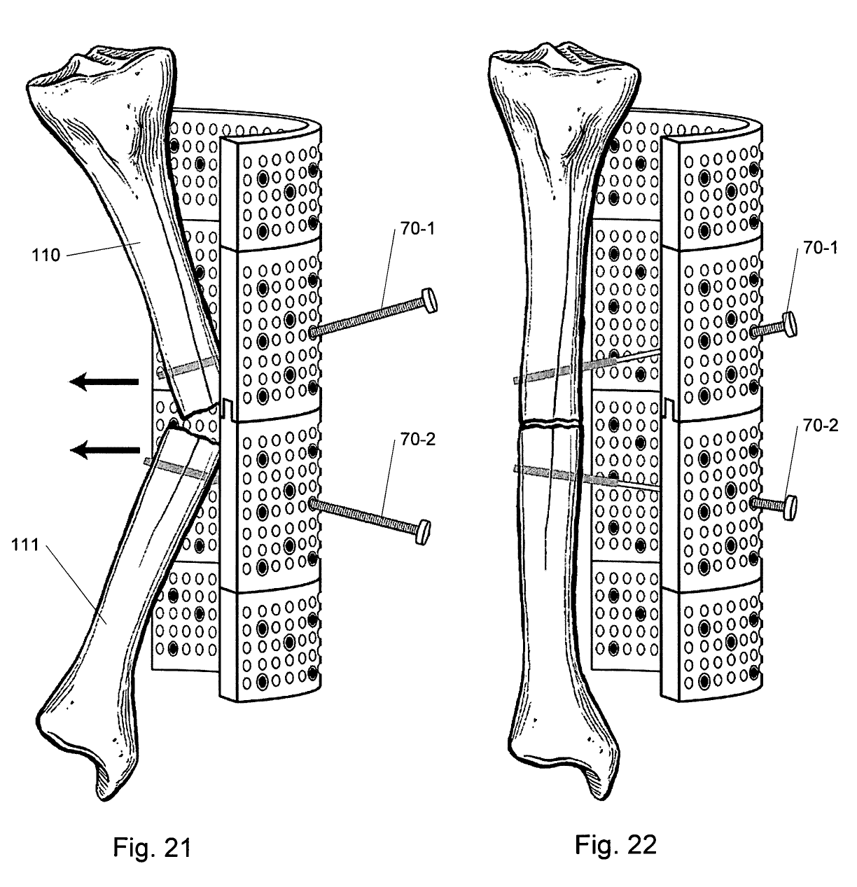

FIGS. 21 and 22 illustrate use of the present apparatus and roller bearing joints 120 to permit half-pins 70-1 and 70-2 to be used to align fractured bone segments 110 and 111 exhibiting angulation. As shown in FIG. 22, half-pins 70-1 and 70-2 are inserted into apertures 52 above and below the point of the fracture and are affixed to the bone fragments at an angle. As each half-pin is advanced toward the bone fragments, in the manner described above, the bone fragments are moved into alignment and the angle between the two fragments reduced. It will be appreciated that as the bone fragments are moved into alignment and away from arc panel 50, the angle of each half-pin changes. The moveable spherical member of roller ball joint 120 accommodates the change in angle and permits the surgeon to achieve a reduction with the margins of the fragmented bones in closer proximity to one another and not overlap.

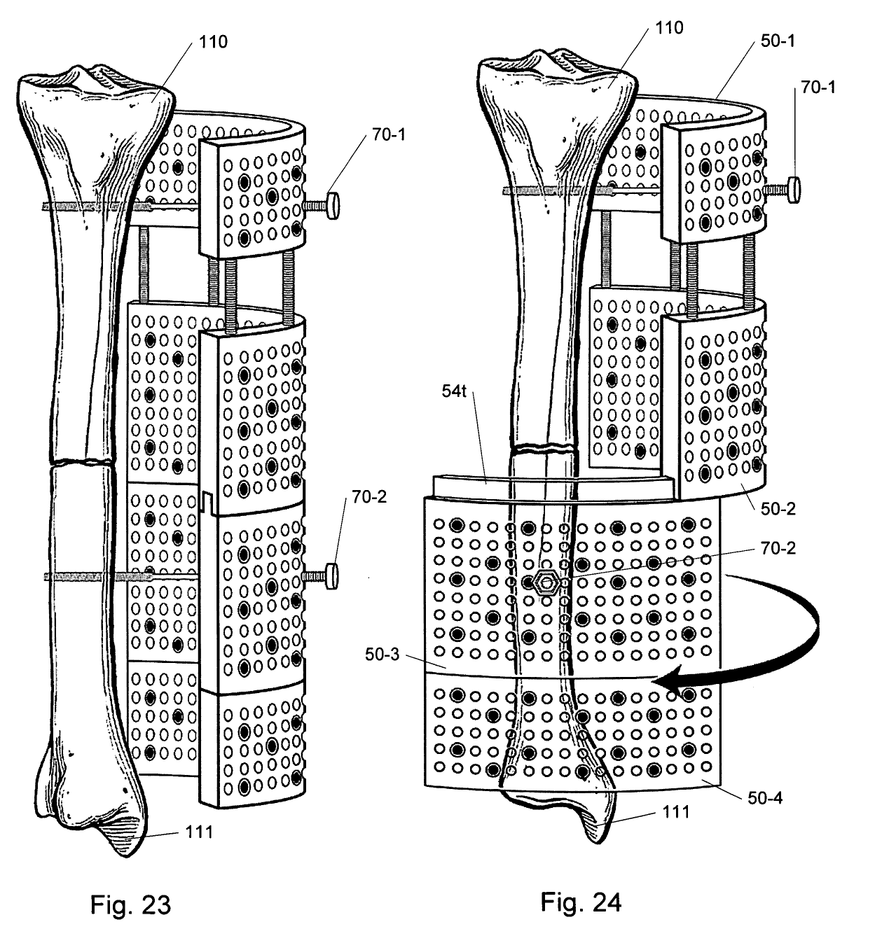

FIGS. 23 and 24 illustrate use of the present apparatus to align fractured bone segments that require rotation with respect to one another in order to reduce the fracture. FIG. 23 illustrates half-pins 70-1 and 70-2 used to affix arc panel 50 to upper and lower fractured bone segments 110 and 111. It will be appreciated that the nature of the fracture illustrated does not require lateral relocation of one fragment with respect to the other, but rather the rotation of one and/or the other bone segment. FIG. 24 illustrates the rotation of first and second arc panel segments 50-1 and 50-2 with respect to lower two segments 50-3 and 50-4 via cooperation of tongue 54t. It is apparent that rotating the two joined segments with respect to one another imparts rotation upon upper and lower bone segments 110 and 111 such that the two bone segments can be aligned. While a simple tongue and groove arrangement is illustrated it is contemplated that more sophisticated constructions, such as rack and pinion, could be used which permit a motorized and/or the computer controlled movement of the arc segments to provide precise and/or automated alignment.

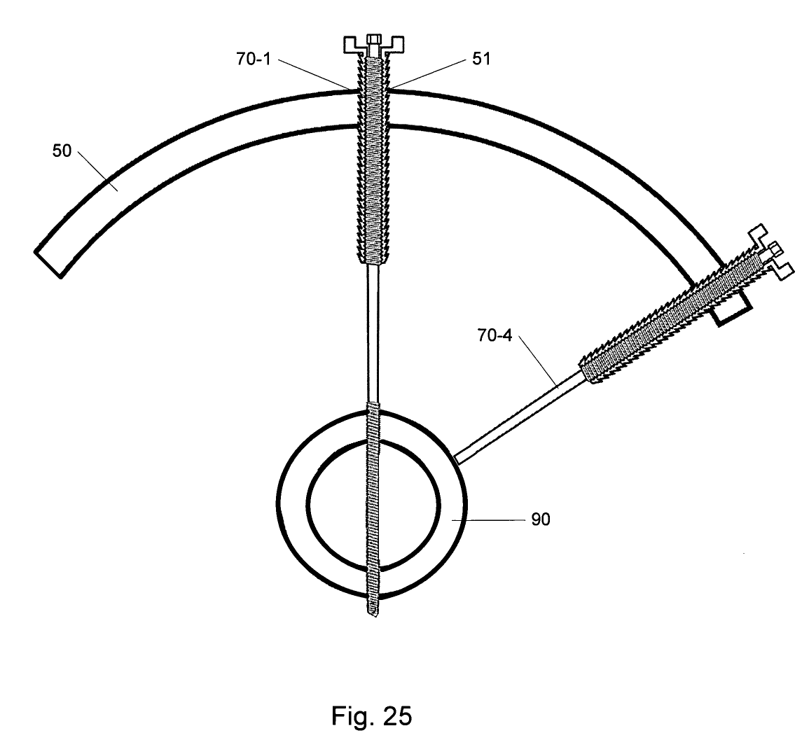

In a further embodiment one or more half-pins can be replaced with blunt end pins, as illustrated in FIG. 25. FIG. 25 of the drawings specifically illustrates a top plan cross-sectional view of the arc fixator panel positioned adjacent a broken limb. One half-pin is secured to the broken bone fragment in a stable manner and one blunt pin 70-4 in positioned in contact with but not affixed to the broken bone fragment. Blunt pin 70-4 is inserted into a pin carrier which is in turn positioned in an aperture within the arc panel.

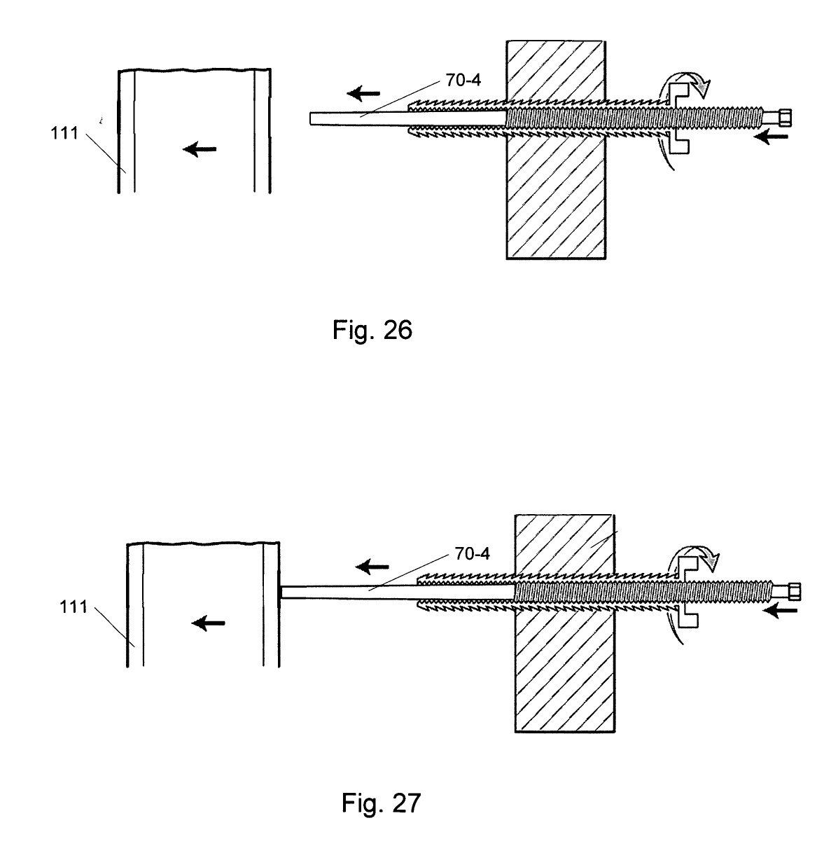

As illustrated in FIG. 26, as the pin carrier is rotated within the arc panel the blunt pin is either advanced or retracted, depending upon the direction or rotation. Accordingly, blunt pin 70-4 can be positioned into contact with the external surface of the bone fragment 111. Further advancing of the blunt pin 70-4 serves to apply force directly upon the bone fragment 111 and, in turn, move the bone fragment in the direction of the applied force without the need to pierce the broken bone fragment, as illustrated in FIG. 27. It will be appreciated that the use of a blunt pin that is not affixed to the bone fragment offers certain benefits that provide the surgeon with greater flexibility in aligning broken fragments using the arc panel of the present invention. It can be further appreciated that the present invention permits a surgeon to work quicker and subject a patient to less trauma and the surgeon to less radiation.

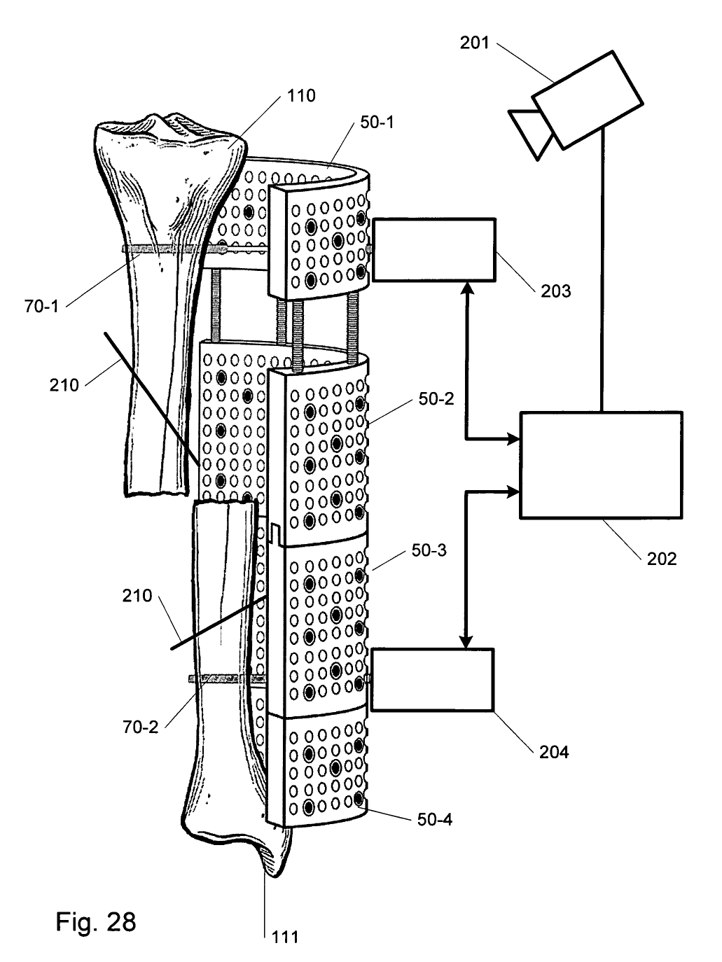

As discussed, it is contemplated that a control system may be provided to synchronize the movement of a single or multiple pin carriers 80, and, in turn, associated half-pins 50 to provide uniform and/or automated control. For example as illustrated in FIG. 28, pin carriers 80 are each connected to electronic servo motors 203 and 204, respectively, such that rotation of each pin carrier is computer controlled and integrated into a fully automated alignment system. This system may optionally rely upon reference wires 210 inserted by the surgeon to the distal and proximal bone fragments which when x-rayed by unit 201 and analyzed by the control computer 202 establish the initial position of the fractured bone fragments. A control computer 202 commands and controls the rotation of pin carriers 80 to advance corresponding half-pins 70 so as to move the fractured bone fragments into alignment all without manual intervention. The system may further operate in an incremental manner where interim x-rays are taken and analyzed to monitor the progress and verify bone fragment position at various steps. Alternatively, the system may initially compute the total distance required to move the bone fragments into position to provide optimum alignment. The control system performs the mathematical computations necessary to determine the degree of movement. Option software analyzes the nature of the fracture whereby multiple alignment jacks may be moved in a coordinated manner to articulate bone fragments into proper alignment. Optional back pressure sensors are integrated into servo motors to monitor the force exerted on the limb and provide limits, warnings or otherwise permit optimum computer control over the movement of the individual bone fragments.

In a further embodiment of the present invention full (llizarov-type) rings can be attached to the upper and lower (most proximal and distal aspects) of arc fixator panel 50 to allow for the use of tensioned wires in the most proximal and distal parts of the fractured bone segments.

The foregoing description and drawings merely explain and illustrate the invention and the invention is not limited thereto, as those skilled in the art who have the disclosure before them will be able to make modifications and variations therein without departing from the scope of the invention.

1. A system for externally repairing fractured bones and facilitating alignment of displaced fractured bone segments without requiring use of an external ring fixator system and tension wires, the system comprising:
at least one panel member having a plurality of apertures therein, extending from a first side of the at least one panel member through to a second side of the at least one panel member;
at least one pin carrier, capable of being inserted into at least one of the plurality of apertures in the at least one panel member;
each pin carrier, upon insertion into at least one of the plurality of apertures in a panel member, being longitudinally fixed relative to the aperture, but capable of rotation within the aperture;
at least two half-pins, at least one half-pin being capable of insertion into at least one pin carrier positioned into one of the plurality of apertures provided in the panel member, toward subsequent securement to a fractured bone segment;
the at least one panel member being further operably configured to accommodate at least one outrigger member capable of being removably affixed to the at least one panel member, the at least one outrigger member being capable of accepting receipt of a half-pin toward securing the outrigger member to a fractured bone segment; the at least two half-pins serving, in part, to support the at least one panel member overlying the fractured bone segments;
whereupon rotation of a pin carrier causes an associated half-pin inserted therein to move longitudinally with respect to the at least one panel member to, in turn, reposition the fractured bone segment affixed to the half-pin, relative to the at least one panel member.

2. The system according to claim 1 wherein the plurality of apertures provided in the panel member are formed in a plurality of symmetrical rows and columns providing multiple points at which a half-pin can be inserted and positioned relative to the bone segments to be aligned.

3. The system according to claim 1 wherein the at least one pin carrier is substantially cylindrical in shape and includes at least one annular ring formed on an outer facing surface thereof.

4. The system according to claim 3 wherein the at least one pin carrier includes a helical thread formed on an inner facing surface thereof which thread cooperates with threads formed on at least a portion of the at least one half-pin.

5. The system according to claim 4 wherein the at least one pin. carrier further includes a control surface to facilitate rotation of the at least one pin carrier, following insertion of the at least one pin carrier into one of the plurality of apertures in the at least one panel member.

6. The system according to claim 5 wherein the control surface comprises a knob.

7. The system according to claim 3 wherein each of the plurality of apertures within the at least one panel member is formed with at least one annular ring on an inner surface thereof, operably configured for cooperation with an outer facing annular ring of the at least one pin carrier.

8. The system according to claim 1 wherein at least one panel member is further provided with apertures dimensioned and threaded to directly accept at least one half-pin, without insertion of an intervening pin carrier.

9. The system according to claim 1 wherein the at least one panel member is arc shaped.

10. The system according to claim 9 wherein the arc shaped panel member is operably configured to generally conform to the shape of a limb surrounding fractured bone segments.

11. The system according to claim 1 wherein the panel member further includes a roller bearing joint positioned in at least one aperture capable of accepting a pin carrier and, in turn, a half-pin to be secured to a fractured bone segment at an angle relative to the surface of the panel member.

12. The system according claim 1, further comprising at least one outrigger member capable of being removably affixed to the at least one panel member, the at least one outrigger member being capable of accepting receipt of a half-pin toward securing the outrigger member to a fractured bone segment.

13. The system according to claim 12 wherein the length of the at least one outrigger, upon attachment to an upper or lower edge region of the at least one panel member, may be alternatively increased or decreased as necessary to position the panel member in an overlying orientation with respect to the fractured bone segment.

14. The system according to claim 1 wherein the at least one panel member comprises at least two panel members, secured together to form a single reconfigurable composite panel member capable of supporting pin carriers and, in turn, half-pins in a manner that permits the half-pins to be secured to the fractured bone segments in a plurality of planes.

15. The system according to claim 14 wherein a first panel member incorporates a groove formed on a lower facing edge thereof, and wherein a second panel member incorporates a tongue formed on an upper facing edge thereof, to thereby facilitate joining the first panel member to the second panel member.

16. The system according to claim 14 wherein a first panel member incorporates a threaded aperture formed on a lower facing edge and wherein a second panel member incorporates a threaded aperture formed on an upper facing edge to thereby facilitate joining one panel member to the another panel member using a threaded rod.

17. The system according to claim 16 wherein a first panel member segment may be rotated and secured relative to a second panel member segment.

18. The system according to claim 1 wherein at least one outrigger member is attached to the at least one panel member, and has a half-pin insertably received therethrough to affix the at least one outrigger member, and in turn, the at least one panel member to a fractured bone segment.

19. The system according to claim 1 wherein the at least one outrigger member is adjustable to vary its length to, in turn, separate and align fractured bone segments.

20. A system for externally repairing fractured bones and facilitating alignment of displaced fractured bone segments without requiring use of an external ring fixator system and tension wires, the system comprising:
at least one panel member having a plurality of apertures therein, extending from a first side of the at least one panel member through to a second side of the at least one panel member;
at least two pin carriers, capable of being inserted into at least two of the plurality of apertures in the at least one panel member;
the at least two pin carriers, upon insertion into at least one of the plurality of apertures in the panel member, being longitudinally fixed relative to the aperture, but capable of rotation within the aperture;
at least two half-pins each capable of insertion into a pin carrier, following insertion of the pin carrier into one of the plurality of apertures provided in the panel member, toward subsequent securement to a fractured bone segment;
whereupon rotation of a pin carrier causes an associated half-pin inserted therein to move longitudinally with respect to the panel member to, in turn, reposition the fractured bone segment affixed to the half-pin, relative to the panel member.

21. The system according to claim 20 wherein the plurality of apertures provided in the panel member are formed in a plurality of symmetrical rows and columns providing multiple points at which a half-pin can be inserted and positioned relative to the bone segments to be aligned.

22. The system according to claim 20 wherein the at least one pin carrier is substantially cylindrical in shape and includes at least one annular ring formed on an outer facing surface thereof.

23. The system according to claim 20 wherein the at least one pin carrier includes a helical thread formed on an inner facing surface thereof which thread cooperates with threads formed on at least a portion of the at least one half-pin.

24. The system according to claim 23 wherein the at least one pin carrier further includes a control surface to facilitate rotation of the at least one pin carrier, following insertion of the at least one pin carrier into one of the plurality of apertures in the at least one panel member.

25. The system according to claim 24 wherein the control surface comprises a knob.

26. The system according to claim 22 wherein each of the plurality of apertures within the at least one panel member is formed with at least one annular ring on an inner surface thereof, operably configured for cooperation with an outer facing annular ring of the at least one pin carrier.

27. The system according to claim 20 wherein at least one panel member is further provided with apertures dimensioned and threaded to directly accept at least one half-pin, without insertion of an intervening pin carrier.

28. The system according to claim 20 wherein the at least one panel member is arc shaped.

29. The system according to claim 28 wherein the arc shaped panel member is operably configured to generally conform to the shape of a limb surrounding fractured bone segments.

30. The system according to claim 20 wherein the panel member further includes a roller bearing joint positioned in at least one aperture capable of accepting a pin carrier, and in turn, a half-pin to be secured to a fractured bone segment at an angle relative to the surface of the panel member.

31. The system according to claim 20 wherein the at least one panel member comprises at least two panel members, secured together to form a single reconfigurable composite panel member capable of supporting pin carriers and, in turn, half-pins in a manner that permits the half-pins to be secured to the fractured bone segments in a plurality of planes.

32. The system according to claim 28 wherein a first panel member incorporates a groove formed on a lower facing edge thereof, and wherein a second panel member incorporates a tongue formed on an upper facing edge thereof, to thereby facilitate joining the first panel member to the second panel member.

33. The system according to claim 28 wherein a first panel member incorporates a threaded aperture formed on a lower facing edge and wherein a second panel member incorporates a threaded aperture formed on an upper facing edge to thereby facilitate joining one panel member to the another panel member using a threaded rod.

34. The system according to claim 28 wherein a first panel member segment may be rotated and secured relative to a second panel member segment.

35. A system for externally repairing fractured bones and facilitating alignment of displaced fractured bone segments without requiring use of an external ring fixator system and tension wires, the system comprising:
at least one panel member having a plurality of apertures therein, extending from a first side of the at least one panel member through to a second side of the at least one panel member;
at least two pin carriers, capable of being inserted into at least two of the plurality of apertures in the at least one panel member;
the at least two pin carriers, upon insertion into at least one of the plurality of apertures in the panel member, being longitudinally fixed relative to the aperture, but capable of rotation within the aperture;
at least one half-pin capable of insertion into a pin carrier, following insertion of the one pin carrier into one of the plurality of apertures provided in the panel member, toward subsequent securement to a fractured bone segment;
at least one blunt pin capable of insertion into a pin carrier, following insertion of the one pin carrier into one of the plurality of apertures provided in the panel member, toward subsequent movement into contact with a fractured bone segment;
whereupon rotation of a pin carrier causes an associated half-pin or blunt pin inserted therein to move longitudinally with respect to the panel member to, in turn, reposition a fractured bone segment relative to the panel member.

36. An automated computer assisted system for aligning displaced fractured bone fragments, the system comprising:
a panel member having a plurality of apertures therein and affixable to the displaced fractured bone fragments by a plurality half-pins with at least a first half-pin positionable above the point fracture and a second half-pin positionable below the point of fracture, the panel member, in turn, secured to the upper and lower fractured bone fragments by half-pins passed through the skin and into underlying bone;
at least one pin carrier positioned in at least one aperture and configured to accept telescopic receipt of a half-pin, the rotation of the pin-carrier serving to adjust the position of the half-pin with respect to the panel member toward facilitating alignment of the displaced fracture bone fragments;
a computer controlled electronic servo motor operably connected to each pin carrier for precisely and independently adjusting the position of a half-pin positioned therein;
computer controlled x-ray imaging and control system for creating and analyzing an electronic image, determining the relative displacement of the fractured bone fragments and signaling the controlled electronic servo motors to adjust the position of each pin carrier toward aligning the displaced fractured bone fragments without manual intervention.

37. The automated computer assisted system for aligning displaced fractured bone fragments according to claim 35 further including reference wires capable of being inserted into the fractured bone fragments to enhance detection of the bone fragments by the x-ray imaging and control system and establish the initial position of the fractured bone fragments.

38. The automated computer assisted system for aligning displaced fractured bone fragments according to claim 36 wherein computer controlled x-ray imaging and control system analyzes the initial position of the fractured bone fragments, computes the distance and sequence in which each bone fragment must be moved in order to properly restore alignment and signals the controlled electronic servo motors in a coordinated manner to move in the proper sequence toward articulating the fractured bone fragments into proper alignment.

39. The invention according to claim 36 wherein the automated computer assisted system further includes back pressure sensors associated with one or more pin carriers and/or half-pins toward monitoring the force exerted by the half-pin upon the limb and providing an alarm signal to the x-ray imaging and control system.

40. A method for aligning displaced fractured bone fragments, the method comprising the steps of:
providing a panel member for affixation to the fractured limb with at least a first half-pin positionable above the fracture and a second half-pin positionable below the fracture;
affixing at least one pin carrier to the panel member positioned adjacent the fracture;
inserting at least one half-pin into at least one pin carrier and affixing the half-pin to a fractured bone fragment;
adjusting the pin carrier to reposition the associated half-pin to reposition the underlying bone fragment affixed thereto to reduce the fracture and align the bone fragments;
whereby the fracture is reduced and bone fragments aligned without the surgeon having to use external ring fixator system and tension wires to reposition misaligned bone fragments.
